(19) United States
(12) Patent Application Publication
(10) Pub. No.: US 2022/0133392 A1
(43) Pub. Date: May 5, 2022

(54) MICROFLUIDIC CHIP FOR ATTRACTING AND DESTROYING A SPECIFIC BIOLOGICAL ELEMENT

(71) Applicants: UNIVERSITE DE MONTPELLIER, Montpellier (FR); CENTRE NATIONAL DE LA RECHERCHE SCIENTIFIQUE (CNRS), Paris (FR); ECOLE NATIONALE SUPERIEURE DE CHIMIE DE MONTPELLIER, Montpellier (FR)

(72) Inventors: Benoît CHARLOT, Montpellier (FR); Jean-Marie RAMIREZ, Usclas-du-Bosc (FR); Sébastien MEANCE, Montpellier (FR); Xavier GARRIC, Montpellier (FR); Coline PINESE, Montpellier (FR); Isabelle GUIRAUD, Saint-Hilaire-de-Brethmas (FR)

(73) Assignees: UNIVERSITE DE MONTPELLIER, Montpellier (FR); CENTRE NATIONAL DE LA RECHERCHE SCIENTIFIQUE (CNRS), Paris (FR); ECOLE NATIONALE SUPERIEURE DE CHIMIE DE MONTPELLIER, Montpellier (FR)

(21) Appl. No.: 17/435,075

(22) PCT Filed: Mar. 12, 2020
(86) PCT No.: PCT/FR2020/050527

§ 371 Date: Aug. 31, 2021

(30) Foreign Application Priority Data
Mar. 15, 2019 (FR) 1902686

(51) Int. Cl. A61B 18/14; A61N 1/40; A61K 38/19; A61K 38/18

(52) U.S. Cl. CPC A61B 18/14; A61N 1/40; A61K 38/195; A61K 38/1841; A61B 2018/00613; A61K 38/1858; A61K 38/1808; A61K 38/1866; A61B 2018/1407; A61K 38/1825

(57) ABSTRACT

The present invention relates to a microfluidic chip for attracting and destroying a specific biological element, said chip comprising:
a reservoir (1) consisting of a matrix comprising a chemoattractant compound capable of attracting the biological element
at least one network of microchannels (2) arranged between the reservoir (1) and the external environment (3) of the chip and allowing the chemoattractant compound to pass to said environment and the biological element present in said environment to pass to the reservoir (1)
at least one electrode (4) arranged between the reservoir (1) and the network of microchannels (2) or at the same location as the network of microchannels (2), said electrode (4) being capable of generating an electric field so as to destroy the biological element during its passage to the reservoir (1).

### TECHNICAL FIELD

The present application relates to the field of microfluidic devices capable of attracting and destroying a specific biological element. More precisely, the present application relates to a microfluidic chip capable of attracting and destroying in vivo a specific biological element, such as a prokaryotic or eukaryotic cell.

### PRIOR ART

Each year in France, roughly 400 000 new cases of cancer are identified, with about 150 000 deaths.

There are different types of cancer depending on the tissue in which they develop. Solid tumors, which are characterized by a localized cluster of cancer cells, are distinct from blood cell tumors, which are diffuse with cancer cells circulating in the bone marrow or blood. Blood cell tumors, also called hematopoietic cancers, are cancers that affect the blood or lymphoid organs, such as leukemia and lymphoma.

Concerning solid tumors, carcinomas are distinct from adenocarcinomas, which are cancers arising from epithelial tissue. Also distinct are sarcomas, which are cancer cells appearing in a so-called support tissue, such as osteosarcoma for bones, liposarcoma for fat and myosarcoma for muscles.

In both men and women, solid cancers represent 90% of cancers, with prostate, lung, and colorectal cancers representing the three most common cancers in men, and breast, colorectal, and lung cancers representing the three most common cancers in women. Although cancer mortality has decreased by 1.5% per year in men and 1% per year in women between 1980 and 2012 (standardized rates), there remains a need for effective solutions to treat and prevent these solid tumors and their complications.

To date, surgery is one of the principal treatments for solid tumors and involves the resection of the entire tumor when possible and, possibly, the tissue around the tumor, called the resection margin. Surgery can be used as a single treatment when the tumor is highly localized, in particular when the tumor is at an early stage, but it is often combined with other treatments such as radiotherapy, which is also a local treatment, and/or medical treatments such as chemotherapy, which is a systemic treatment potentially acting on all of the body's cancer cells.

The advantage of local surgery for the treatment of a solid tumor is the ability to remove the entire tumor when possible and to preserve organs and anatomical structures not affected by the cancer cells. It also limits the side effects attributed to radiotherapy, such as burns or the generation of radio-induced cancers, and to chemotherapy, such as skin reactions, nausea, vomiting, diarrhea, muscle pains, fatigue, hair loss as well as chemo-induced cancers.

In order to reduce the rate of recurrence, the resection area includes an area of healthy tissue around the tumor, which is the resection margin. At this stage, several scenarios must be considered. In the first scenario, the tumor cells of the primary tumor are included in the resection area and there will be no recurrence. In the second scenario, some tumor cells are located beyond the resection area, either locally or at a distance, and then a recurrence is possible, either locally or at a distance, to form metastases, i.e., secondary colonies of cancer cells that spread at a distance from the organ affected by the initial tumor and that are at the origin of a so-called “metastatic” cancer in an organ other than the one in which the solid tumor was located.

It is therefore necessary to prevent as much as possible the risk of local recurrence and metastatic cancer after resection of a solid tumor.

Chemotherapy can be used after local surgery to resect the tumor, referred to as “adjuvant chemotherapy” to prevent recurrence and/or the formation of metastatic cancer. However, as indicated above, numerous side effects are associated with this drug treatment. Among the most significant side effects related to the use of chemotherapy, particular reference may be made to chemo-induced cancers, which are by definition new tumors arising in patients treated with cytotoxic drugs for a first malignant tumor and caused by the latter, reference may also be made to the risk of generating a resistance of cancer cells to the chemotherapeutic treatment, thus strongly limiting the possibilities of eliminating said cells.

Thus, there is no solution to date to effectively eliminate the remaining cancer cells after resection of a solid tumor in order to prevent and/or decrease the risk of local recurrence and/or development of metastatic cancer, which is effective and does not present short and medium term drug side effects.

To respond to these needs, the present invention proposes a microfluidic chip for attracting and destroying a specific biological element and in particular a eukaryotic cell such as a cancer cell. Said chip being particularly suitable to be implanted in vivo at the resection area of a solid tumor in order to attract and destroy in vivo the remaining cancer cells.

Microfluidic devices have been described in the prior art for use in the field of cancer treatment. Particular reference may be made to WO2018/089989 A1 which describes an ex vivo device for the treatment of cancer by subjecting a biological fluid such as blood to electromagnetic radiation specific to the type of cancer cell being targeted and capable of destroying it.

Reference may also be made to document US2018111124 A1, which describes a microfluidic device comprising one or more microfluidic channels and one or more wireless bipolar electrode arrays enabling high-throughput capture of tumor cells circulating in a conductive ionic solution by applying a 40 kHz alternating electric field to a biological sample. The cells thus captured can be used to diagnose cancer or evaluate the effect of cancer treatment.

No prior art document describes or suggests a microfluidic chip for attracting and destroying, preferably in vivo, a cancer cell after resection of a solid tumor and thus preventing and/or decreasing the risks of local recurrence and/or development of metastatic cancer.

More generally, no prior art document describes or suggests a microfluidic chip for attracting and destroying, preferably in vivo, a specific biological element, said microfluidic chip comprising a reservoir consisting of a matrix comprising a chemoattractant compound capable of attracting a biological element, at least one network of microchannels putting the reservoir and the external environment of the chip into communication, and at least one electrode capable of generating an electric field for destroying the biological element.

### DISCLOSURE OF THE INVENTION

The present invention relates to a microfluidic chip for attracting and destroying a specific biological element, said chip comprising:
a reservoir (1) consisting of a matrix comprising a chemoattractant compound capable of attracting the biological element
at least one network of microchannels (2) arranged between the reservoir (1) and the external environment (3) of the chip and allowing the chemoattractant compound to pass to said environment and the biological element present in said environment to pass to the reservoir (1)
at least one electrode (4) arranged between the reservoir (1) and the network of microchannels (2) or at the same location as the network of microchannels (2), said electrode (4) being capable of generating an electric field so as to destroy the biological element during its passage to the reservoir (1).

### BRIEF DESCRIPTION OF THE FIGURES

FIG. 1 is a cross-sectional view of the chip in accordance with the invention.

FIG. 2 shows the upper part (6) of the chip seen from above.

FIG. 3 shows the lower part (5) of the chip seen from above.

FIG. 4 shows an exploded view of the bottom of the chip in accordance with the invention.

FIG. 5 shows an exploded view of the top of the chip in accordance with the invention.

FIG. 6 shows a cross-sectional view of the chip in accordance with the invention at a scale of 5:0.5 cm.

FIG. 7 shows a cross-sectional view of the chip at a scale of 5:1 cm.

FIG. 8 is an inverted fluorescence microscope image with a 5× objective, at the level of the microchannels, showing breast cancer tumor cells (MDA-MB-231) (14) in the absence of gradient.

FIG. 9 is an inverted fluorescence microscope image with a 5× objective, at the level of the microchannels, showing breast cancer tumor cells (MDA-MB-231) (14) drawn into the chip from a gradient of fetal calf serum (FCS) with 1% FCS outside the chip and 10 μl of pure FCS inside the reservoir.

FIG. 10 is an inverted fluorescence microscope image with a 10× objective, at the level of the microchannels, showing breast cancer tumor cells (MDA-MB-231) (14) in the absence of gradient.

FIG. 11 is an inverted fluorescence microscope image with a 5× objective, at the level of the central reservoir of the chip, showing breast cancer tumor cells (MDA-MB-231) (14) in the absence of gradient.

FIG. 12 is an inverted fluorescence microscope 10× objective image, at the level of the microchannels, showing breast cancer tumor cells (MDA-MB-231) (14) drawn into the chip from a gradient of fetal calf serum (FCS) with 1% FCS outside the chip and 10 μl of pure FCS inside the reservoir.

FIG. 13 is an inverted fluorescence microscope 5× objective image, at the level of the central reservoir of the chip, showing breast cancer tumor cells (MDA-MB-231) (14) drawn into the chip from a gradient of fetal calf serum (FCS) with 1% FCS outside the chip and 10 μl of pure FCS inside the reservoir.

FIG. 14 is an inverted fluorescence microscope 10× objective image, at the level of the microchannels, showing breast cancer tumor cells (MDA-MB-231) (14) drawn into the chip from a gradient of chemoattractant SDF-1 (1 μg of chemoattractant SDF-1 present in the central reservoir of the chip).

FIG. 15 is an inverted fluorescence microscope 5× objective image, at the level of the central reservoir of the chip, showing breast cancer tumor cells (MDA-MB-231) (14) drawn into the chip from a gradient of chemoattractant SDF-1 (1 μg of chemoattractant SDF-1 present in the central reservoir of the chip).

FIG. 16 is an inverted fluorescence microscope image, showing breast cancer tumor cells (MDA-MB-231) on a glass slide containing an interdigitated electrode, after 48 h of culture in an incubator with 5% CO₂ and 95% humidity, in the absence of electroporation.

FIG. 17 is an inverted fluorescence microscope image of breast cancer tumor cells (MDA-MB-231) on a glass slide containing an interdigitated electrode, destroyed by electroporation through the application of an electric field by an interdigitated electrode of 5 V 100 μs 1 hertz at d0 and at d0+ 3 hours.

FIG. 18 is an inverted fluorescence microscope image, at the level of the microchannels, showing breast cancer tumor cells (MDA-MB-231) in the absence of gradient and of pulsed electric field.

FIG. 19 is an inverted fluorescence microscope image, at the level of the microchannels, showing breast cancer tumor cells (MDA-MB-231) drawn into the chip from an SDF-1 gradient (1 μg of chemoattractant SDF-1 present in the central reservoir of the chip) without a pulsed electric field.

FIG. 20 is an inverted fluorescence microscope image, at the level of the microchannels, showing breast cancer tumor cells (MDA-MB-231) drawn into the chip from an SDF-1 gradient (1 μg of chemoattractant SDF-1 present in the central reservoir of the chip) and a pulsed electric field of 5 V 100 ms 1 hertz.

FIG. 21 is a graph showing the release of BSA-FITC (mimicking SDF-1) from an alginate matrix contained in a microfluidic chip in accordance with the invention over a period of 50 days.

FIG. 22 is a graph showing the release of SDF-1 from an alginate matrix over a period of 50 days.

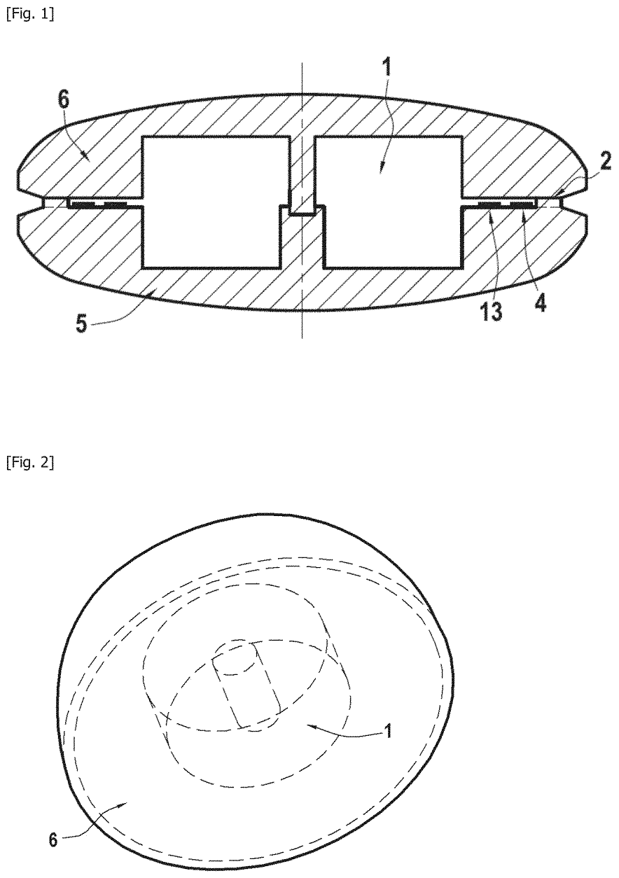

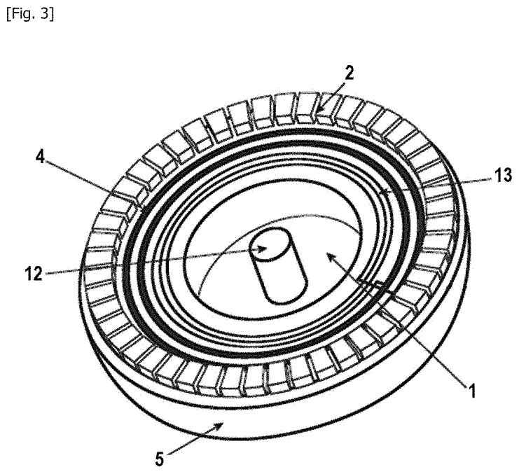

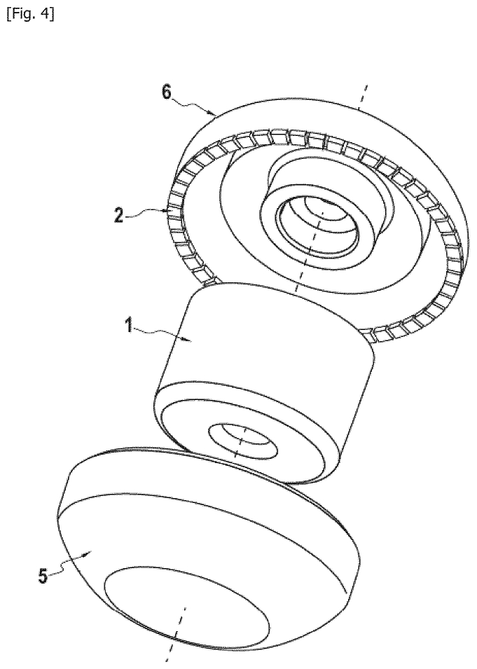

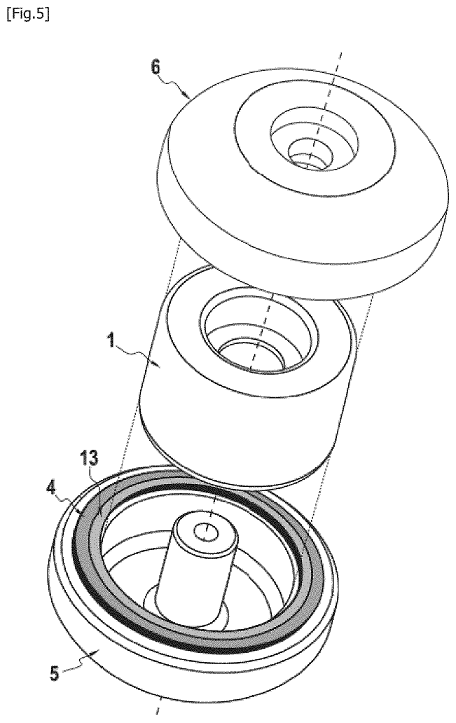

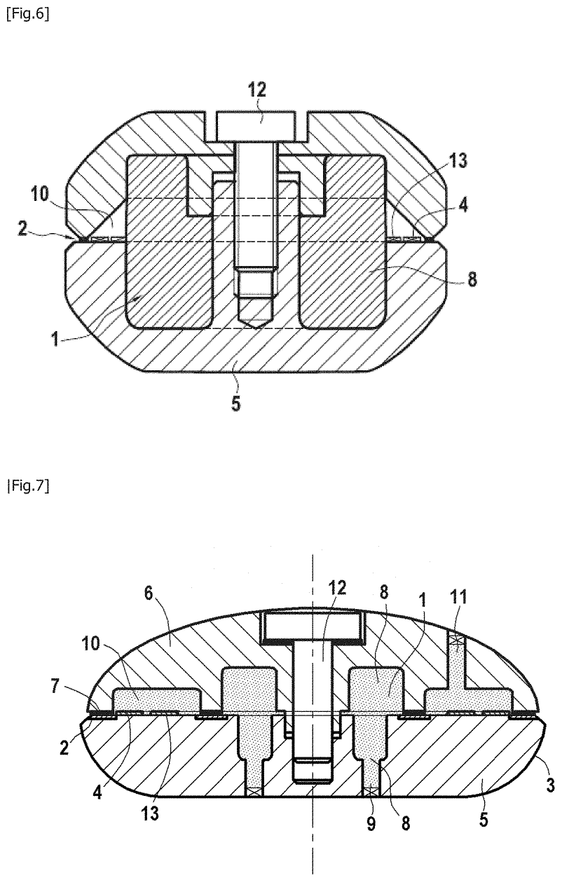

### DETAILED DESCRIPTION

The Applicants have developed a microfluidic chip to attract and destroy a biological element, such as a prokaryotic or eukaryotic cell. This microfluidic chip is particularly advantageous for attracting and destroying a cancer cell in vivo after the resection of a solid tumor in order to prevent and/or decrease the risks of local recurrence and/or development of metastatic cancer.

### Definitions

A “microfluidic chip” is a device comprising a network of microchannels, i.e., channels of micrometer size, etched or molded in a material, connected to each other and linking the inside of the chip to the outside the chip by means of inlets and outlets drilled through the chip, in order to perform a desired function. The microfluidic chip can be obtained by specific processes such as deposition and electrodeposition, etching, bonding, injection molding, embossing, soft lithography, anodic soldering, or any other technologies. These manufacturing processes are known to the person skilled in the art. In the context of the present invention, the desired function of the microfluidic chip is to be able to attract in vivo a specific biological element, preferably a eukaryotic cell such as a cancer cell, and to destroy it in vivo.

The “network of microchannels” corresponds to a multitude of channels, connected to the outside the chip by inlets and outlets drilled through the chip. The microchannels can for example be fabricated from a mold, or directly in the material of the microfluidic chip. The number of microchannels varies according to the diameter of the chip, the width of the microchannels or the spacing between said microchannels. By way of example, for a chip with a diameter of one centimeter and a channel width of 10 μm, the number of microchannels can be roughly 1500.

Each microchannel composing the network of microchannels corresponds to a passage whose height can be from a few micrometers to a few hundred micrometers with a length of a few hundred micrometers up to a few millimeters. The cross-section of a microchannel can in principle have any two-dimensional shape, such as a square, a rectangle, a circle or a combination thereof. A microchannel can be straight or curved.

The width of a microchannel is the horizontal distance of the two points that are on opposite edges of the cross-section and are farthest apart. The height of a microchannel is the vertical distance of the points on opposite edges of the cross-section that are farthest apart. The length of a microchannel is the distance between the two ends of said channel, the length of a microchannel corresponds to the largest dimension. The two shorter dimensions generally define the aforementioned cross-section.

In the context of the present invention, “electrode” refers to any element capable of conducting an electric current and comprising two conductors separated by an air gap of a distance between 1 μm and 1 mm. The present application refers to an electrode, in the singular for the sake of clarity, the skilled person understanding that an electrode is composed of said two conductors separated by an air gap so that the current can be conducted.

The microfluidic chip is preferably “intended to be implanted in vivo”, i.e., the microfluidic chip is intended and capable of being implanted within a living being, preferably a mammal and in particular a human being. More particularly, this means that the chip can be implanted in a living being without interfering with or degrading the tissues with which it is in contact and that said chip is capable of functioning in vivo, i.e., attracting and destroying a specific biological element in vivo, preferably a eukaryotic cell such as a cancer cell, and destroying it in vivo.

In the sense of the present invention, “biological element” refers to any element comprising genetic information in the form of RNA or DNA and likely to be found within a living organism, i.e., in vivo, such as prokaryotic cells, eukaryotic cells and microorganisms. Among the conceivable biological elements, particular reference may be made to a prokaryotic cell such as a bacterium, to a eukaryotic cell such as an animal cell.

“Specific biological element” or “target biological element” refers to the biological element of interest in the context of the use of the chip, i.e., the biological element intended to be attracted and destroyed in vivo. The chemoattractant compound present in the reservoir is selected to attract the biological element of interest in the context of the use of the microfluidic chip.

“At the same location as the network of microchannels” refers to the fact that the electrode is arranged at the level of the network of microchannels, more precisely on or under the network of microchannels. Preferably, the electrode is arranged on the network of microchannels.

Preferably in the context of the invention, the biological element of interest is a prokaryotic or eukaryotic cell, more preferably the biological element of interest is a eukaryotic cell. Even more preferably, the eukaryotic cell is a cancer cell, preferably a metastatic cancer cell.

A “cancer cell” is a cell in which one or more major DNA lesions have occurred, thereby transforming the normal cell into a cancer cell capable of proliferating to form a group of identical transformed cells, i.e., a tumor. In particular, a cell is referred to as a cancer cell when the cell in question has a number of features such as independence from cell growth regulation signals, an ability to escape the process of programmed cell death and an ability to divide indefinitely.

More particularly in the context of the present invention, “cancer cell” refers to a cell from a so-called initial or original or primary solid tumor, present at or near the solid tumor resection area.

Preferably in the context of the present invention, “cancer cell” also refers to a “metastatic cancer cell”, i.e., a cancer cell capable of or having migrated through the body via blood or lymphatic vessels from the original tumor and capable of or having colonized one or more other tissues in the vicinity of or at a distance from said tumor, thus forming metastases, at the origin of “metastatic cancers” or also “metastatic tumors”. In this context, the cancer cell is a cell originating from a so-called secondary or tertiary solid tumor, which correspond to metastatic tumors in a second or third tissue or organ other than that of the initial tumor.

Reference is made to “specific biological element”, “a prokaryotic cell”, “a eukaryotic cell”, “a cancer cell”, “a metastatic cell” in the singular for the sake of clarity, it being understood that the microfluidic chip in accordance with the present invention is capable of attracting and destroying in vivo several of said elements and cells. It is also good to note that the microfluidic chip in accordance with the present invention is capable of attracting and destroying in vivo several specific biological elements which may be of different nature, said elements being drawn into the chip by the specific choice of chemoattractant compound(s) present in the reservoir.

In the context of the present invention, the term “prevent” means a reduction in the risk of acquiring a specified disease or disorder, the reduction or the slowing of the onset of symptoms of that disease. For example, in the context of the present invention, the term “prevent” may correspond to the reduction of the risk of spreading an infection when the biological element is a prokaryotic cell or to the reduction of the risk of local recurrence of cancer and/or the risk of the appearance of metastasis, more specifically metastatic cancers, when the biological element is a eukaryotic cell of the cancerous type.

In the context of the present invention, the term “treat” means an improvement or reversal of a specified disease or disorder or at least one discernible symptom. The term “treat” may also mean the reduction or the slowing of the progression of the disease or disorder, or the onset of symptoms of the disease or disorder. For example, in the context of the present invention, the term “treat” may correspond to the reduction or the slowing of the progression of an infection when the biological element is a prokaryotic cell, or to the reduction or the slowing of the appearance of metastasis, more specifically metastatic cancers, when the biological element is a eukaryotic cell of the cancerous type.

In the sense of the present invention, the microfluidic chip is preferably intended to be implanted in vivo in a subject.

The subject in the context of the present invention is a living being, preferably a mammal and more particularly human beings, children, men or women.

“Solid cancer” or “solid tumor” refers to an individualized mass of cancer cells in any tissue such as skin, mucous membranes, bone, or any other tissue present in the organs, i.e., carcinomas originating from epithelial cells such as skin, mucous membranes, glands and sarcomas originating from cells of connective and supporting tissues such as bone, cartilage.

Preferably in the context of the present invention, “solid cancer” or “solid tumor” refers to a carcinoma such as cancer of the breast, lung, prostate, bladder, salivary glands, skin intestine, colon-rectum, thyroid, cervix, endometrium and ovaries, lip-mouth-larynx, kidney, liver, brain, testicles, pancreas, preferentially breast cancer. These examples of solid tumors are non-limiting.

“Chemoattractant compound” refers to any compound capable of attracting by chemotaxis a biological element, preferably a cell expressing specific membrane receptors of this compound on its surface, said biological element moving as a function of the concentration gradient of chemoattractant compound. In the context of the present invention, the chemoattractant compound is capable of inducing the displacement of one or more specific biological elements as a function of the concentration gradient of said compound by positive chemotaxis, with the biological element moving to the region where the concentration of chemoattractant compound is highest.

In particular in the context of the present invention, the chemoattractant compound is said to be “suitable for attracting” a specific biological element when it allows the movement of said element within the microfluidic chip and in particular towards the chemoattractant compound reservoir in which the highest concentration of chemoattractant compound is found. The skilled person will know how to characterize the specific biological element of interest in order to choose the chemoattractant compound suitable for attracting said element within the chip.

In the context of the present invention, the chemoattractant compound is selected based on the specific biological element of interest.

When the biological element is a eukaryotic cell, the chemoattractant compound is selected according to the type of membrane receptors that this cell expresses.

More precisely, when the biological element is a eukaryotic cell, the chemoattractant compound can be a cytokine, i.e., a polypeptide or a soluble protein synthesized by a cell and acting at a distance on other cells to regulate their activity and function via membrane receptors, selected from chemokines, granulocyte and macrophage colony stimulating factors such as M-CSF, G-CSF, CSF-1, growth factors and transforming growth factors such as TGF alpha, TGF beta, EGF, betacellulin, amphiregulin, heregulin, HBEGF, FGF, VEGF, tumor necrosis factors such as NGF, TNF alpha, TNF beta, interferons such as IFN alpha, IFN beta, IFN gamma, IFN lambda and interleukins such as IL-1 to IL-38.

Preferably when the biological element is a eukaryotic cell such as a cancer cell, the chemoattractant compound is selected from chemokines, growth factors and transforming growth factors.

A chemokine is a small protein of 8 to 14 kilodaltons characterized by the presence of four cysteine residues in conserved positions allowing the formation of their three-dimensional structure. Chemokines can be classified into four subfamilies according to the spacing between two of their cysteines in the N-terminal position, particular reference may be made to the CXC or alpha family, i.e., whose first two cysteines are separated by any amino acid, the CC or beta family, the CX3C or delta family and the C or gamma family. The chemokine in the context of the present invention may for example be selected from the following chemokines: CXCL12, also called stromal cell-derived factor 1 (SDF-1), CCL5, CCL2, CCL3, CCL7, CCL19, CCL21, CCL22, CCL25, CXCL1, CXCL5, CXCL6, CXCL8, CX3CL1.

A growth factor is a low-molecular-weight protein (less than 30 kilodaltons) that stimulates cell multiplication and is recognized by specific membrane receptors which are most often tyrosine kinases. The growth factor in the context of the present invention may for example be selected from TGF alpha or beta (transforming growth factor alpha or beta), FGF (fibroblast growth factor alpha), EGF (epidermal growth factor), betacellulin, amphiregulin, heregulin, HBEGF, VEGF (vascular endothelial growth factor), PDGF (platelet-derived growth factor).

When the biological element is a prokaryotic cell, such as a bacterium, the chemoattractant compound can be a peptide bearing a formylated N group such as N-formylmethionyl-leucyl-phenylalanine (FMLP) or carbohydrate molecules such as glucose.

In the context of the present invention, the chemoattractant compound is comprised in a matrix that is composed of a biocompatible material as defined in the present invention. The biocompatible material of the matrix being selected specifically as a function of the chemoattractant compound, the desired release profile as well as the context of use of the microfluidic chip.

In the context of the present invention, “external environment” refers to the tissues located around the microfluidic chip when the latter is implanted in vivo, more precisely to the tissues located directly in contact with the chip, up to 300 mm, preferably up to 150 mm, even more preferably up to 100 mm around the chip.

“Resection of a solid tumor” means the removal, ablation or excision of a solid tumor, for example by surgery.

Thus, the present invention relates to a microfluidic chip for attracting and destroying in vivo a specific biological element.

Numerical references used in the context of the present detailed description of the invention refer to the figures of the present application having the objective of illustrating the invention without being limited thereto.

A first subject matter of the invention relates to a microfluidic chip for attracting and destroying a specific biological element, said chip comprising:
a reservoir (1) consisting of a matrix comprising the chemoattractant compound capable of attracting a biological element
at least one network of microchannels (2) arranged between the reservoir (1) and the external environment (3) of the chip and allowing the chemoattractant compound to pass to said environment and the biological element present in said environment to pass to the reservoir (1)
at least one electrode (4) arranged between the reservoir (1) and the network of microchannels (2) or at the same location as the network of microchannels (2), said electrode being capable of generating an electric field so as to destroy the biological element during its passage to the reservoir (1).

More precisely, the present invention relates to a microfluidic intended to be implanted in vivo to attract and destroy a specific biological element, preferably a eukaryotic cell, said chip comprising:
a reservoir (1) consisting of a matrix comprising the chemoattractant compound capable of attracting a biological element in vivo
at least one network of microchannels (2) arranged between the reservoir (1) and the external environment (3) of the chip and allowing the chemoattractant compound to pass to said environment and the biological element present in said environment to pass to the reservoir (1)
at least one electrode (4) arranged between the reservoir (1) and the network of microchannels (2) or at the same location as the network of microchannels (2), said electrode being capable of generating an electric field so as to destroy the biological element in vivo during its passage to the reservoir (1).

According to a preferred aspect, the present invention relates to a microfluidic intended to be implanted in vivo to attract and destroy a eukaryotic cell, preferably a cancer cell, said chip comprising:
a reservoir (1) consisting of a matrix comprising the chemoattractant compound capable of attracting said cell in vivo
at least one network of microchannels (2) arranged between the reservoir (1) and the external environment (3) of the chip and allowing the chemoattractant compound to pass to said environment and said cell present in said environment to pass to the reservoir (1)
at least one electrode (4) arranged between the reservoir (1) and the network of microchannels (2) or at the same location as the network of microchannels (2), said electrode (4) being capable of generating an electric field so as to destroy said cell during its passage to the reservoir (1) in vivo.

According to a first particular embodiment, the present invention relates to a microfluidic chip intended to be implanted in vivo for attracting and destroying a specific biological element, preferably a eukaryotic cell, more preferably a cancer cell, said chip comprising:
a reservoir (1) consisting of a matrix comprising the chemoattractant compound capable of attracting said cell in vivo
at least one network of microchannels (2) arranged between the reservoir (1) and the external environment (3) of the chip and allowing the chemoattractant compound to pass to said environment and said cell present in said environment to pass to the reservoir (1)
at least one electrode (4) arranged between the reservoir (1) and the network of microchannels (2), said electrode (4) being capable of generating an electric field so as to destroy said cell during its passage to the reservoir (1) in vivo.

According to a second particular embodiment, the present invention relates to a microfluidic chip intended to be implanted in vivo for attracting and destroying a specific biological element, preferably a eukaryotic cell, more preferably a cancer cell, said chip comprising:
a reservoir (1) consisting of a matrix comprising the chemoattractant compound capable of attracting said cell in vivo
at least one network of microchannels (2) arranged between the reservoir (1) and the external environment (3) of the chip and allowing the chemoattractant compound to pass to said environment and said cell present in said environment to pass to the reservoir (1)
at least one electrode (4) arranged at the same location as the network of microchannels (2), said electrode (4) being capable of generating an electric field so as to destroy said cell during its passage to the reservoir (1) in vivo.

Preferably in this embodiment, the electrode is arranged on or under the network of microchannels, more preferably the electrode is arranged on the network of microchannels.

The matrix of the reservoir (1) comprising the chemoattractant compound is formed of a biocompatible material.

The chemoattractant compound comprised in the matrix of the reservoir (1) attracts in vivo the specific biological element within the chip by positive chemotaxis, the biological element migrating to the reservoir (1) where the concentration of chemoattractant compound is the highest. The network of microchannels (2) allows the chemoattractant compound to diffuse from the reservoir (1) to the external environment (3) of the chip, as well as the specific biological element to migrate from the external environment (3) of the chip to the inside of the chip, to the reservoir (1). The electrode (4) is arranged between the reservoir (1) and the network of microchannels (2) such that when the specific biological element enters the chip from the external environment (3) through the network of microchannels (2) and to the reservoir (1), the electric field generated by said electrode (4) destroys the biological element in vivo during its passage to the reservoir (1).

In the context of the present invention, the biological element is preferably a cancer cell and in particular a cancer cell originating from a cancer or a solid tumor. The migration of this cell inside the chip is carried out by adhesion to the support on which the electrode (4) is arranged, the electric field generated by the electrode being then able to destroy the cell in vivo during its passage to the reservoir (1).

The network of microchannels (2) of the microfluidic chip in accordance with the present invention is preferably located on the outer edge of said chip, i.e., in contact with the external environment (3), thus ensuring communication between said environment and the interior space of the chip.

The chip in accordance with the present invention may comprise several networks of microchannels (2), for example two networks of microchannels. According to a preferred aspect, the microfluidic chip in accordance with the invention comprises a single electrode (4). According to another aspect, the microfluidic chip in accordance with the invention comprises several electrodes (4).

According to particular embodiments of the present invention, the electrode may be more particularly arranged:
on both sides of at least one network of microchannels,
within at least one network of microchannels,
between at least two networks of microchannels or
adjacent to at least one network of microchannels.

The microfluidic chip in accordance with the first subject matter of the invention comprises a lower part (5) and an upper part (6), the reservoir (1) comprising the chemoattractant compound matrix, the network of microchannels (2) and the electrode (4) being able to be comprised independently of each other in the upper part (6) and/or the lower part (5) of said chip.

The upper part (6) of the microfluidic chip is suitable to be arranged on the lower part (5) to form a cover, said parts being fixed to each other.

According to a particular aspect, the microfluidic chip in accordance with the present invention comprises a lower part (5) comprising a part of the reservoir (1), the network of microchannels (2) and the electrode (4), and an upper part (6) comprising a part of the reservoir (1) and suitable to be arranged on the lower part (5), said parts being fixed to each other.

According to another particular aspect, the microfluidic chip in accordance with the present invention comprises a lower part (5) comprising a part of the reservoir (1), the electrode (4), and an upper part (6) comprising a part of the reservoir (1) and the network of microchannels (2), said upper part being suitable to be arranged on the lower part (5), said parts being fixed to each other.

With regard to the electrode (4), “comprised in” means that the electrode is deposited and/or integrated on the lower and/or upper part of the chip. Preferably, the electrode is integrated on the lower part of the chip.

The reservoir (1) comprised in the lower part (5) and in the upper part (6) is a single reservoir, one part of which is in the upper part of the chip and the other part in the lower part of the chip.

In the context of the present invention, the expression upper part (6) “suitable for being arranged on the lower part (5) to form a cover” means that the shape of the upper part (6) is such that it fits the shape of the lower part (5) on which it rests without hindering the functionality of each element composing the lower part (5) and allows the formation of a cover closing the chip.

Advantageously and preferably, the upper (6) and lower part (5) of the microfluidic chip are rounded so that the chip can be implanted in vivo without damaging the tissue.

Preferably the chip is rounded in shape, with for example the upper and lower part having a half-oval or half-sphere shape so that when the upper part is arranged on the lower part, the microfluidic chip is oval or spherical in shape, respectively.

The size of the microfluidic chip in accordance with the first subject matter is suitable for in vivo implantation and is of the order of a few centimeters, preferably between 0.5 and 5 cm, more preferably between 1 and 3 cm, more preferably 1 cm.

More particularly, the present invention relates to a microfluidic chip in which the reservoir (1), the network of microchannels (2) and the electrode (4) are ring-shaped and in which the reservoir is located in the center of the chip.

As its name indicates, the ring shape corresponds to the shape of a ring. This particular configuration ensures in particular a radial diffusion of the chemoattractant compound contained in the reservoir (1) to the external environment (3) and a homogeneous attraction of the biological element from said environment to the reservoir (1), thanks to the central location of the reservoir (1) as well as to the ring shape of the network of microchannels (2).

Similarly, the ring shape of the electrode (4) arranged between the reservoir (1) and the network of microchannels (2) advantageously ensures effective destruction of each biological element that penetrates the chip from any direction.

The lower part (5) and the upper part (6) of the microfluidic chip can be fixed to each other by any physical or chemical means (12), suitable for in vivo use. By way of example of a physical or chemical fixing means, respective reference may be made to a screw or an adhesive suitable for in vivo use of the chip or to a protruding element coming opposite a hollow element present in the lower and upper part to join them.

Preferably in the context of the present invention, the upper part (6) comprises one or more openings through which one or more screws can be inserted and the lower part (5) comprises one or more nuts suitable for receiving said screw(s).

Even more preferably, the upper part (6) of the microfluidic chip in accordance with the invention comprises a central opening through which a screw can be inserted and the lower part (5) comprises a central nut capable of receiving said screw. In this preferred embodiment, the ring-shaped reservoir (1) is arranged around the central opening present on the upper part (6) and the central nut present on the lower part (5) of the chip.

In particular, the microfluidic chip comprises one or more seals (7) capable of sealing the chip, said seals (7) being arranged between the lower part (5) and the upper part (6) above the network of microchannels (2).

“Seal capable of sealing the chip” refers to the property of the seal not to allow fluids that may be present in the external environment (3), such as blood, to enter the chip, and not to allow liquids and materials present inside the chip to leave the chip through any other location than the network of microchannels (2). The position of the seal(s) (7) above the network of microchannels (2) does not hinder the diffusion of the chemoattractant compound to the external environment (3) nor the passage of the specific cell present in said environment within the chip, to the reservoir (1). Advantageously, the seals create a support area between the upper and lower part of the chip.

In particular, the present invention relates to a microfluidic chip in which the upper part (6) and/or the lower part (5) comprise a ring-shaped cavity i) (8) capable of receiving the matrix comprising the chemoattractant compound, and a ring-shaped cavity ii) (10) arranged between the reservoir (1) and the network of microchannels (2), capable of receiving a liquid in which the chemoattractant compound is capable of diffusing.

More particularly, the present invention relates to a microfluidic chip in which the upper part (6) and/or the lower part (5) comprise a ring-shaped cavity i) (8) capable of receiving the matrix comprising the chemoattractant compound via one or more openings (9) communicating with the external environment (3) and opening into said cavity, and a ring-shaped cavity ii) (10) arranged between the reservoir (1) and the network of microchannels (2), suitable for receiving a liquid into which the chemoattractant compound is capable of diffusing, by means of one or more openings (11) communicating with the external environment (3) and opening into said cavity.

More particularly still, the present invention relates to a microfluidic chip in which the upper part (6) and the lower part (5) comprise a ring-shaped cavity i) (8) capable of receiving the matrix comprising the chemoattractant compound via one or more openings (9) communicating with the external environment (3) and opening into said cavity and a ring-shaped cavity ii) (10) arranged between the reservoir (1) and the network of microchannels (2), suitable for receiving a liquid into which the chemoattractant compound is capable of diffusing, by means of one or more openings (11) communicating with the external environment (3) and opening into said cavity.

Preferably the chemoattractant compound is added to the reservoir matrix (1) prior to its addition into the ring-shaped cavity i) (8). Alternatively, the chemoattractant compound may be added to the ring-shaped cavity i) (8) before or after the addition to the same cavity of the reservoir matrix (1).

The matrix comprising the chemoattractant compound and the liquid into which the chemoattractant compound is capable of diffusing are added to the ring-shaped cavity i) (8) and the ring-shaped cavity ii) respectively through said openings (9, 11), preferably after the upper part (6) has been placed on the lower part (5).

The liquid into which the chemoattractant compound is capable of diffusing is preferably an aqueous solution such as saline or a physiological buffer solution such as an aqueous solution comprising a phosphate-buffered saline (PBS).

The impermeability of each of the cavities after said additions is ensured by closing the openings through seals, for example made of PDMS or any other suitable material, said seals being located at the outlet of these openings (9, 11) and communicating with the external environment (3).

Preferably, the ring-shaped cavity i) (8) is comprised in the upper part (6) and the lower part (5) of the chip, the opening(s) (9) leading into this cavity being located in the lower part (5) of the chip, and the ring-shaped cavity ii) (10) as well as the opening(s) (11) leading into this cavity are comprised in the upper part (6) of the chip.

The chip in accordance with the present invention is made of biocompatible material. In particular, the present invention relates to a microfluidic chip in which the upper part (6) and the lower part (5) are composed of a biocompatible material.

More precisely, the upper part (6) and the lower part (5) as such as well as the elements they comprise are made of a biocompatible material. More precisely still, the upper part (6), the lower part (5), the reservoir (1), in particular the matrix comprising the chemoattractant compound, the network of microchannels (2), the electrode (4) and the seals (7) are made of a biocompatible material.

“Biocompatible material” or “biomaterial” refers to a material that does not interfere with or degrade the biological environment in which it is used, even in direct or indirect, brief or sustained contact with the internal tissues and fluids of the body of a human being or animal. By way of example of biocompatible material that can be used in the context of the present invention, non-exhaustive reference may be made to glass, ceramics such as alumina, zirconia, hydroxyapatite, metals and metal alloys such as titanium, platinum, polymers of natural origin such as collagen, agarose, chitosan, carrageenan, xanthan and alginate, or degradable synthetic polymers such as polyesters and polyanhydrides, or non-degradable polymers such as polyurethanes, cellulose and its derivatives, vinyl polymers. Preferably in the context of the present invention the polymers of synthetic origin are PEEK (polyetheretherketone) or PDMS (polydimethylsiloxane).

Preferably, the upper part (6), the lower part (5) and the network of microchannels (2) are independently of each other made of polymers of synthetic origin such as polydimethylsiloxane (PDMS) or polyetheretherketone (PEEK). Even more preferably, the upper part (6), the lower part (5) are made of polyetheretherketone (PEEK).

Preferably, the network of microchannels (2) is made of polyetheretherketone (PEEK).

Preferably, the electrode (4) is made of titanium and/or platinum, more preferably of titanium and platinum.

Preferably, the reservoir (1) and in particular the matrix comprising the chemoattractant compound is made of collagen and/or alginate, more preferably said reservoir, in particular said matrix is made of alginate.

In a particular and preferred manner, the upper part (6), the lower part (5) and the network of microchannels (2) of the chip in accordance with the invention are made of polyetheretherketone (PEEK), the reservoir (1) of chemoattractant compound is made of alginate and the electrode (4) is made of titanium and platinum.

According to a particular aspect, the present invention relates to a microfluidic chip in which the matrix comprising the chemoattractant compound is composed of a biocompatible material, preferably a crosslinked polymer. Preferably, the crosslinked polymer composing the matrix is a polymer of natural origin and in particular collagen and/or alginate, preferably alginate.

The crosslinking of a polymer corresponds to the formation of one or more three-dimensional networks from linear or branched polymers, by chemical and/or physical means. The person skilled in the art knows how to induce a crosslinking of polymers according to the polymers considered, as an example the crosslinking can be carried out by heating and/or by the use of crosslinking agent. A “crosslinked” polymer is a polymer in which some of its chains are linked together by strong or weak bonds.

By way of example, collagen can be crosslinked using crosslinking agents such as ammonia gas, oxidized sugars or aldehydes at room temperature and alginate can be crosslinked in a calcium chloride bath at room temperature.

Even more preferably, the biocompatible material composing the matrix comprising the chemoattractant compound is alginate crosslinked in a calcium chloride bath preferably at room temperature.

The crosslinking of the biocompatible material composing the matrix comprising the chemoattractant compound advantageously allows a sustained release of this compound. “Sustained release” means a controlled and continuous release kinetics of the chemoattractant compound over a period of time. Preferably in the context of the present invention, the release of the chemoattractant compound occurs between 3 days and 6 months, preferably between 15 days and 3 months.

According to a particular aspect, the present invention relates to a microfluidic chip in which the mass percentage of chemoattractant compound/matrix of the reservoir (1) is comprised between 0.1 and 20%, preferably between 0.5 and 10% and more preferably between 0.5 and 5%.

The person skilled in the art will know how to determine the content of the reservoir in chemoattractant compound depending on the context of the use of the chip, the specific biological element of interest, the desired release time of the chemoattractant compound and the biomaterial composing the reservoir matrix.

When the specific biological element is a eukaryotic cell, preferably a cancer cell, the chemoattractant compound comprised in the reservoir of the microfluidic chip and in particular in the reservoir matrix is preferably selected from chemokines such as CXCL12, also called stromal cell-derived factor 1 (SDF-1), CCL5, CCL2, CCL3, CCL7, CCL19, CCL21, CCL22, CCL25, CXCL1, CXCL5, CXCL6, CXCL8, CX3CL1, growth factors and transforming growth factors such as TGF alpha or beta (transforming growth factor alpha or beta), FGF (fibroblast growth factor alpha), EGF (epidermal growth factor alpha), betacellulin, amphiregulin, heregulin, HBEGF, PDGF (platelet-derived growth factor), VEGF (vascular endothelial growth factor).

More particularly, the present invention relates to a microfluidic chip in which the chemoattractant compound comprised in the reservoir (1) of the chip, is selected from at least one of the following compounds: CXCL12, CCL5, CCL2, CCL3, CCL7, CCL19, CCL21, CCL22, CCL25, CXCL1, CXCL5, CXCL6, CXCL8, CX3CL1, TGF alpha, TGF beta, FGF, PDGF, EGF, VEGF.

According to another aspect, when the specific biological element is a prokaryotic cell, preferably a bacterium, the chemoattractant comprised in the reservoir (1) of the microfluidic chip and in particular in the matrix of the reservoir (1), is preferably selected among carbohydrate molecules.

The chemoattractant compound can be used alone or in combination with one or more of the other chemoattractant compounds mentioned above and/or with other compounds capable of directly or indirectly improving the ability of said chemoattractant compound to attract a specific biological element such as a eukaryotic cell and in particular a cancer cell, such as for example carbohydrate (glucose) and/or lipid (fatty acid) molecules which make it possible to provide the necessary energy (energy provided in the form of ATP after degradation of glucose or fatty acids) for the survival of the biological element, in particular of a eukaryotic cell and in particular of a cancer cell. Other molecules such as oxygen can be used in association with chemoattractants. Oxygen can be transported by hemoglobin or by synthetic hemoglobins.

Oxygen is an essential molecule for the survival and proliferation of cells, especially cancer cells. Preferably the chemoattractant compound is used in combination with one or more carbohydrate (glucose) and/or lipid (fatty acid) molecules.

The skilled person will take care to choose the chemoattractant compound(s) according to the specific biological element targeted.

To this end, when the biological element is a eukaryotic cell, an analysis of the membrane receptors expressed by the targeted cell must be carried out beforehand to ensure the specificity of the chemoattractant compound(s) selected.

By way of example, when the specific biological element is a cancer cell expressing the transmembrane receptor CXCR4, such as a human breast cancer cell, the chemoattractant compound selected is stromal cell-derived factor 1 (SDF-1).

Similarly, when the specific biological element is a cancer cell from lung cancer, the chemoattractant compound selected is epidermal growth factor (EGF) or transforming growth factor alpha (TGF alpha).

The microchannels composing the network of microchannels (2) of the microfluidic chip in accordance with the present invention may be parallelepipedic, cylindrical, squamous, frustoconical or a mixture of these shapes.

Each microchannel comprised in the network of microchannels (2) of the microfluidic chip in accordance with the present invention may have a height comprised between 1 and 500 μm, preferably between 50 μm and 150 μm, a width comprised between 1 and 500 μm, preferably between 50 μm and 150 μm, and a length comprised between 30 μm and 1 mm, preferably between 30 μm and 500 μm.

In particular in the context of the invention, each microchannel comprised in the network of microchannels (2) has a height comprised between 1 and 40 μm, a width comprised between 1 and 40 μm, a length comprised between 30 and 250 μm.

Preferably in the context of the present invention, each microchannel comprised in the network of microchannels (2) has a height comprised between 5 and 20 μm, a width comprised between 5 and 20 μm, and a length comprised between 100 and 200 μm, preferably 200 μm.

Each microchannel comprised in a network of microchannels may have its own dimensions independently of the dimensions of the other microchannels comprised in the network of microchannels.

Similarly, when the chip comprises several networks of microchannels, each microchannel of the same network as well as each microchannel comprised in the different networks of microchannels can have its own dimensions and shapes independently of each other.

Preferably, in the context of the present invention all the microchannels comprised in a network of microchannels have the same shape and the same dimensions.

According to a particular aspect, the present invention relates to a microfluidic chip in which the electrode (4) is an interdigitated electrode.

The “interdigitated electrode” corresponds to an electrode (4) in which each conductor is multi-toothed. Preferably each conductor has a diameter comprised between 200 and 400 μm, preferably about 315 μm, and each conductor is spaced from the other by an air gap comprised between 10 and 50 μm, preferably about 15 μm.

Even more preferably, the electrode (4) is a wireless electrode, remotely powered by an antenna (13).

For example, the remote power supply can be operated by wireless communication, for example from the ISM (industrial, scientific and medical) frequency band using a frequency range comprised between 13.553-13.567 MHz.

More particularly, the present invention relates to a microfluidic chip in which the electrode (4) is a wireless electrode, said chip comprising an antenna activatable by a second antenna. Preferably the second antenna is intended to be placed ex vivo when the microfluidic chip is intended to be implanted in vivo.

“Antenna” refers to an electronic component consisting of a winding of a conductive material forming one or more turns. When this antenna is crossed by a current, it produces a magnetic field. This magnetic field can be released by the antenna as electrical energy. The antennas thus allow the transmission of electricity through the tissue by inductance. Preferably, in the context of the present invention, the antenna is an inductor.

The antenna (13) allows the remote power supply of the electrode (4).

The antenna can also allow the transmission of information remotely.

The second antenna to be located ex vivo has a transmitter function and the antenna comprised in the wireless electrode a receiver function.

The person skilled in the art will take care to adapt the resonance frequency, the distance, and the alignment between the transmitter and the receiver according to the efficiency of the wireless power to be transmitted.

The antenna comprised in the chip as well as the second antenna are made of a biocompatible material, preferably the same as the one the electrode (4) is made of, which is preferably titanium.

Even more particularly, when the chip is intended to be implanted in vivo, the diameter of said antennas is sufficient for the transmission of electricity through the tissues by inductance, preferably the diameter of the antennas is of the same order of magnitude as the thickness of the tissues to be traversed, preferably the diameter of said antennas is comprised between 5 mm and 100 mm, even more preferably between 10 and 80 mm.

According to a preferred aspect, the second antenna is integrated into a patch intended for topical application to the skin, said patch comprising a physiologically acceptable carrier for topical application, i.e., compatible with the skin, mucous membranes and skin appendages.

The second antenna can also be located in an external box.

According to a particular aspect, the present invention relates to a microfluidic chip in which the electric field generated by the electrode (4) is capable of destroying the biological element by irreversible electroporation.

Preferably, said electric field generated by the electrode is a pulsed electric field, which corresponds to a selective non-thermal treatment of short duration, typically a few microseconds to a few milliseconds.

The application of a pulsed electric field on a specific cell causes the accumulation of charges on the membrane surface and the increase of the transmembrane potential of the cell membrane. The attraction between the charges of opposite signs accumulated on both sides of the cell membrane causes a compression of the latter with an elastic force tending to oppose this electrocompression. When the pulsed electric field applied exceeds a critical value, the electrocompressive force becomes greater than the elastic force, and pores appear in the cell membrane. When the intensity of the pulsed electric field is high and/or the duration of the treatment is long, there is an intensification of the permeabilization and an irreversible destruction of the cell membrane.

In the context of the present invention, the pulsed electric field generated by the electrode on the specific biological element induces irreversible electroporation of said biological element. More precisely, when the biological element is a prokaryotic or eukaryotic cell, the pulsed electric field generated by the electrode creates pores, in particular nanopores in the cell membrane, inducing an irreversible deregulation of cell homeostasis resulting in the death of the cell, by apoptosis or necrosis.

More particularly, the electric field generated by the electrode (4) is a pulsed electric field between 1000 V/cm and 6500 V/cm of voltage, preferably between 3000 V/cm and 5000 V/cm, 1 Hz of frequency and a duration between 100 μs and 200 μs.

Preferably, the pulsed electric field with a duration comprised between 100 μs and 200 μs is generated by the electrode several times a day, preferably at least 3 times, for a period of time appropriate to the context of use of the chip.

According to a particular embodiment of the invention, the microfluidic chip comprises a means for measuring the number of specific biological elements entered into the microfluidic chip and/or the number of biological elements destroyed by the electrode.

Among the conceivable means, particular reference may be made to a system of measurement by electrical impedance via the electrode generating the electrical field destroying the biological element or by other electrodes dedicated to said measurement and which can be comprised in the upper and/or lower part of the microfluidic chip.

Electrical impedance is the measure of the system's opposition to the movement of electrical charges when a potential difference is applied thereto. In other words, it corresponds to the ratio of the voltage applied to the system and the resulting electric current. In the context of the present invention, this measurement is used to quantify the specific biological elements that enter the microfluidic chip, their adhesion to the support inducing a change in electrical impedance, as well as to quantify the specific biological elements destroyed by the electrode, which detach from the electrode and consequently induce a change in electrical impedance.

Preferably, the means for measuring the number of biological elements entered into the microfluidic chip and/or biological elements destroyed by the electrode is an electrical impedance measurement means. In this particular embodiment of the invention, the information can be collected and transmitted by wireless communication technology, for example from the ISM (industrial, scientific and medical) frequency band using a frequency range comprised between 13.553-13.567 MHz.

A second subject matter of the invention relates to the use of the microfluidic chip in accordance with the first subject matter of the invention to attract and destroy a specific biological element.

More particularly, the present invention relates to the use of the microfluidic chip in accordance with the first subject matter of the invention or a method for attracting and destroying a specific biological element in vivo, said chip being implanted in vivo.

More precisely, the present invention relates to the use of the microfluidic chip in accordance with the first subject matter of the invention or a method for treating or preventing the proliferation and dissemination of a specific biological element in a subject, such as a prokaryotic or eukaryotic cell in which the chip is implanted in vivo in a subject.

According to one aspect, the present invention relates to the use of the microfluidic chip in accordance with the first subject matter of the invention or a method for treating or preventing an infection caused by a prokaryotic cell such as a bacterium in which the chip is implanted in vivo in a subject.

According to a preferred aspect, the present invention relates to the use of the microfluidic chip in accordance with the first subject matter of the invention or a method for treating or preventing the proliferation and dissemination of a eukaryotic cell and in particular a cancer cell, preferably after resection of a solid tumor in a subject, wherein the chip is implanted in vivo in a subject.

More precisely, the present invention relates to the use of the microfluidic chip in accordance with the first subject matter of the invention or a method for preventing the risks of local recurrence of cancer and/or development of metastatic cancer in a subject, wherein the chip is implanted in vivo in a subject.

In the context of these uses and methods, the microfluidic chip is implanted at a distance of 0.1 to 20 cm, preferably 1 to 10 cm, more preferably at a distance of 5 cm from the resection area of a solid tumor or the focus of bacterial infection in a subject. The microfluidic chip is preferably implanted at the level of the resection area as soon as the solid tumor is removed, preferably immediately after the resection of said tumor.

In the context of these uses and methods, the pulsed electric field is preferably generated by the electrode several times per day, preferably at least 3 times per day, more preferably 8 times per day, over a period of time appropriate to the context of use of the chip.

“Context of use of the chip” refers to the type of specific biological element targeted, i.e., the type of bacterial infection when said element is a bacterium, or the type of cancer cell targeted and in particular the type of solid tumor removed and the stage of said tumor when said element is a cancer cell.

Depending on said context, the pulsed electric field can be generated over a period of 3 days to 6 months, preferably from 2 weeks to 4 months, in the form of a treatment that can be repeated in cycles with or without a rest period.

For example, if the removed solid tumor was at an advanced stage, the pulsed electric field is generated by the electrode at least 3 times per day, preferably 8 times per day, over a period comprised between 8 and 16 weeks. For example, if the removed solid tumor was at an advanced stage, the pulsed electric field is generated by the electrode at least 8 times per day, over a period of 8 to 16 weeks.

In the context of the above uses, the microfluidic chip can be used alone or in combination with the simultaneous or sequential administration of other drug compounds such as anti-cancer compounds, including chemotherapeutic and/or hormonal and/or immunotherapeutic and/or targeted therapy compounds and/or radiation therapy when the specific biological element is a cancer cell.

Finally, the present invention also relates to a microfluidic chip in accordance with the first subject matter for use in attracting and destroying a specific biological element in vivo, said chip being implanted in vivo, in accordance with the aforementioned implementation conditions.

More precisely, the present invention relates to a microfluidic chip in accordance with the first subject matter for use in treating or preventing the proliferation and dissemination of a specific biological element in a subject, such as a prokaryotic or eukaryotic cell, in which the chip is implanted in vivo in a subject in accordance with the aforementioned implementation conditions.

According to one aspect, the present invention relates to a microfluidic chip in accordance with the first subject matter for use in treating or preventing an infection caused by a prokaryotic cell such as a bacterium, wherein the chip is implanted in vivo in a subject.

According to a preferred aspect, the present invention relates to a microfluidic chip in accordance with the first subject matter for use in treating or preventing proliferation and dissemination of a eukaryotic cell and in particular a cancer cell, preferably after resection of a solid tumor in a subject, wherein the chip is implanted in vivo in a subject in accordance with the aforementioned implementation conditions.

Even more preferably, the present invention relates to a microfluidic chip in accordance with the first subject matter for use in preventing the risk of local recurrence of cancer and/or development of metastatic cancer in a subject, wherein the chip is implanted in vivo in a subject in accordance with the aforementioned implementation conditions.

In the context of these uses and methods, when the specific biological element is a eukaryotic cell and in particular a cancer cell, said cell preferably comes from a cancer of the breast, lung, prostate, bladder salivary glands, skin, intestine, colon-rectum, thyroid, cervix, endometrium and ovaries, lip-mouth-larynx, kidney, liver, brain, testicles, pancreas, preferably breast cancer. These examples of solid tumors being non-limiting.

The invention will be further illustrated by the following figures and examples. However, these examples and figures should in no way be construed as limiting the scope of the invention.

### EXAMPLES

### Example 1: Fabrication of the Microfluidic Chip for the In Vitro Concept Study

To perform the in vitro proof of concept, a microfluidic chip containing in its center a reservoir in which a chemoattractant is placed.

a) Integration of Microelectrodes

A glass substrate (dimension 50×50 mm) is prepared in a so-called “piranha” solution (H₂SO₄:H₂O₂, 3:1) in order to clean it and to increase its hydrophilic properties, followed by a dehumidification for 15 minutes at 150° C. on a hot plate to promote the adhesion of the negative photosensitive resin (AZ® nLOF 2020 from MicroChemicals)

The resin is deposited on the substrate by centrifugal coating (3000 rpm, 30 s) to obtain a thickness of 2 μm. The solvents are evaporated by annealing for one minute at 110° C. on a hot plate. In order to initiate crosslinking in some parts of the photoresist, the sample is subjected to UV exposure (70 mJ/cm²). This activation will modify the local properties of the resin which, after baking, will be soluble or not in a solvent. The resin being negative, the part exposed to UV rays will become solid while the other part will dissolve during the development. To continue the crosslinking reaction initiated by the UV exposure, the plate is then subjected to a post-exposure bake (PEB) to provide the energy necessary to complete the process for 1 min at 110° C. Finally, the development will allow to dilute the non-crosslinked parts of the resin in a tetramethyl-ammonium-hydroxide based solvent (TMAH, AZ® 726 MIF from MicroChemicals).

A titanium/platinum (20/200 nm) deposit is then sputtered followed by a so-called “lift-off” (acetone and NANO™ REMOVER PG from MicroChemicals) to remove the resin residue. At the end of this step, the electrodes are structured on the glass plate.

To ensure electrical insulation on the parts that should not be conductive, a micro-structuring of silicon nitride (Si₃N₄) is performed. To this end, the lithography process described above is repeated with an additional alignment step. The deposition of 200 nm of Si₃N₄ is obtained in a PECVD (plasma-enhanced chemical vapor deposition) frame. The areas not protected by the resin are then etched by a reactive ion etching process (ICP RIE). At the end of the process, the device is cleaned with REMOVER PG from MicroChemicals and an air plasma.

b) Fabrication of the Mold in MicroChemicals SU-8 Photosensitive Epoxy Resin

A silicon plate (diameter 76.2 mm) is prepared in a “piranha” solution followed by dehumidification for 15 to 150° C. The SU-8 resin is deposited on the silicon plate by centrifugal coating (3000 rpm, 30 s) to obtain the desired thickness (10 μm or 100 μm depending on the type used SU-8 2010 or SU-8 2100). Solvent evaporation is obtained with very light heating and cooling ramps to minimize mechanical stress in the resin (5° C./min). The sample is subjected to a 365 nm UV exposure of 125 mJ/cm² for a thickness of 10 μm, and 250 mJ/cm² for a thickness of 100 μm. The plate is then subjected to a post-exposure bake (PEB) (4 min at 95° C. for a thickness of 10 μm and 30 min at 95° C. for a thickness of 100 μm). Finally, the development makes it possible to dilute the uncrosslinked parts of the SU-8 resin in a solvent (“SU-8 developer” composed mainly of PGMEA (propylene glycol monomethyl ether acetate)). At the end of this step, the patterns allowing the structuring of the microchannels remain on the silicon plate.

c) Structuring of the Microchannels and Final Assembly of the Chip

Following fabrication of the mold, a polydimethylsiloxane (PDMS) silicone polymer is prepared by mixing it with its curing agent (Sylgard™184 silicone elastomer kit from Dow, ratio 1:10). Bubbles formed during mixing are removed using a desiccator and vacuum pump. Once the PDMS is degassed, it is poured onto the SU-8 mold placed in a petri dish and placed in an oven at 80° C. for at least 2 hours to complete the crosslinking process. After cooling, the PDMS is peeled off and cut with a circular blade to obtain the cylindrical shapes of the devices. These are then pierced with a punch to form the central well that will contain the chemoattractants. The last step consists in gluing the PDMS on a glass substrate to encapsulate the channels. This is achieved by activating the surface to transform the Si—CH₃ function of the PDMS into Si—OH using an O₂ or air plasma generator. Upon contact with the glass (SiO₂), a permanent

Si—O—Si covalent bond will be created.

d) Electrical Instrumentation

The chip is equipped with interdigitated electrodes (distance: 15 μm) imposing a sufficiently intense field (2000 to 5000 V/cm) to cause irreversible electroporation of specific cells and thus induce apoptosis or necrosis of cells that have penetrated the system. A square wave signal (voltage/frequency/high time: 3 to 7.5 V/1 Hz/100 μs) is delivered by a function generator (TG2511A TTi) and visualized by an oscilloscope (TBS1032B Tektronix) during on-chip application. The second channel of the oscilloscope is connected to a high-precision shunt resistor (0.1 W, LVR01R1000FE70 from Vishay) producing an image of the electric current flowing through the electrodes (4). In order to automate the experiment, the instruments are connected by a USB cable to a computer (Raspberry Pi 3 Model B+, operating system: Linux Raspbian 9 (Stretch)). The “virtual instrument software architecture” (VISA) communication is ensured by the “open source” library PyVISA and a Python script is regularly executed by the crontab program of the operating system.

e) Manufacturing of the Release Matrix

A 3% (w/v) aqueous alginate solution was deposited in a porous mold having the shape of the chip reservoir. The mold is immersed in a calcium chloride crosslinking bath for 24 h. After 3 washes with Milli-Q water, the matrix is frozen at −20° C. and then freeze-dried.

Then, the matrix is gently placed in the chip.

### Example 2: Study of Cytokine Release Profile

(A) To protect it from enzymatic proteolysis in vitro and in vivo, the chemoattractant compound SDF-1 is usually complexed to BSA (Bovine Serum Albumin) at a ratio of 1.51 molecules of BSA to 10 molecules of SDF-1. BSA is a large calf serum protein of 6 kDa while SDF-1 is a small cytokine of 8 kDa. As the release of SDF-1 from the matrix is by diffusion, the molecule with the largest hydrodynamic radius will have the greatest impact on the diffusion of the complex: i.e., BSA. That is why, as a first step, this model was used to study the release profile of the SDF-1 compound from a microfluidic chip fabricated according to Example 1. In order to maximize the detection of nanoscale concentrations, we chose to detect BSA coupled to fluorescein isothiocyanate (FITC) by high-performance liquid chromatography (HPLC) coupled to a fluorescence detector.

Sample Preparation:
Deposition of 3.2 μg of BSA-FITC on the freeze-dried alginate matrix contained in the chip
Incubation 30 min at 37° C.

Study Start:
Immersion of the chip in 1.5 ml of PBS in an airtight box
Incubation under shaking at 37° C. for the duration of the study, i.e., 50 days.
Sampling at different times of 750 μl of release solution which will be analyzed by HPLC (water/acetonitrile).

The column used is a column designed to detect proteins such as BSA-FITC.
750 μl of new PBS is added to the release medium to compensate for the previous sample.

Results:

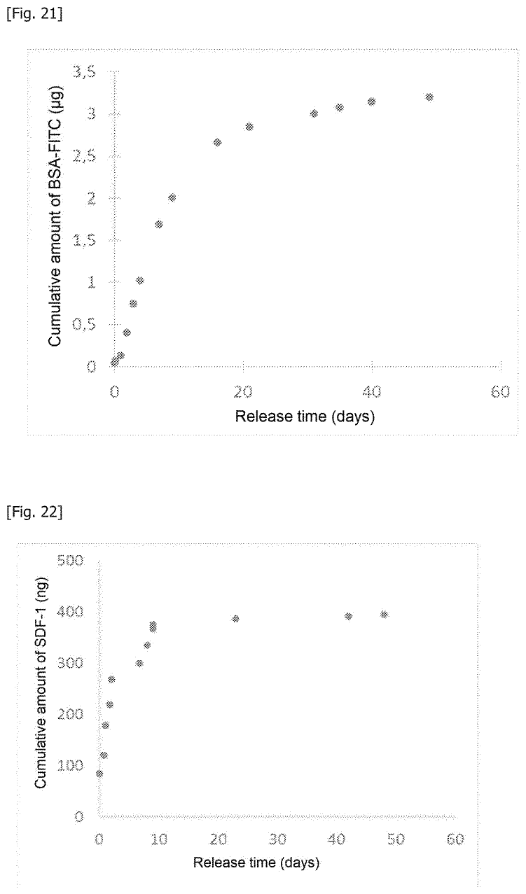

This study demonstrated a sustained release of BSA-FITC from an alginate matrix comprised in a microfluidic chip in accordance with the invention for 50 days with 99% release achieved after 50 days (FIG. 21).

(B) In a second step, the release of SDF-1 from an alginate matrix was quantified over a period of 50 days using an ELISA detection method.

Sample Preparation:
Complexation of SDF-1 with BSA (0.1%)
Deposition of 1 μg of SDF-1 on the freeze-dried alginate matrix
Incubation 30 min at 37° C.

Study Start:
Immersion of the matrix in 1.5 ml of PBS in an airtight box
Incubation under shaking at 37° C. for the duration of the study, i.e., 50 days.
Samples of 750 μl of release solution are taken at different times and analyzed by an

ELISA kit specific for the detection of SDF-1, according to the supplier's instructions.
750 μl of new PBS is added to the release medium to compensate for the previous sample.

Results:

This study demonstrated a sustained release of SDF-1 from the alginate matrix alone for 23 days with 40% release achieved after 23 days (FIG. 22).

### Example 3: Implementation of the Microfluidic Chip In Vitro

A) Capacity of Attraction of the Cells Inside the Chip

The microfluidic chip was implemented in vitro to test its ability to attract MDA-MB-231 breast cancer cells, which are epithelial cells of breast tumors. The compound “stromal cell-derived factor” SDF-1 is the chemoattractant compound capable of attracting MDA-MB-231 cells.

1) MDA-MB-231 cells stably transfected with green fluorescent protein (GFP) are trypsinized at DO. Then 20 000 cells are seeded in a 35 mm petri dish containing the microfluidic chip in its center. The cells are cultured in Dulbecco's Modified Eagle Medium/Nutrient Mixture F-12 (DMEM F12)+Glutamax+1% fetal calf serum (FCS)+1% antibiotic (streptomycin, penicillin).

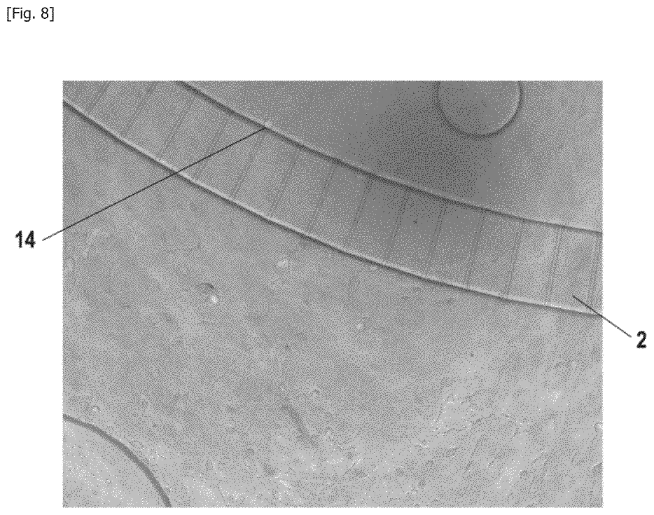

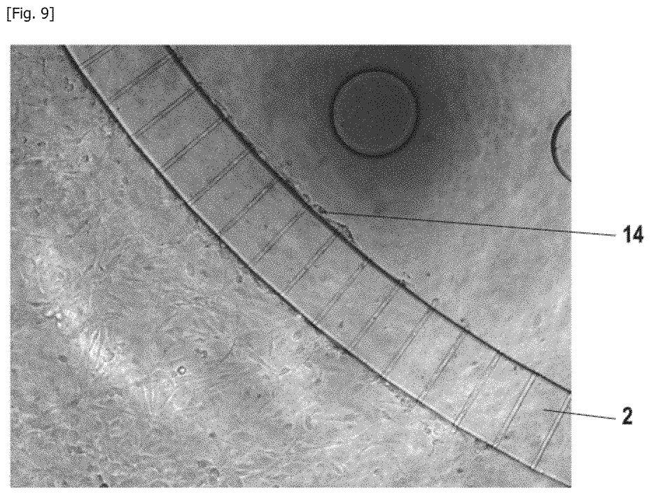

The central reservoir of the microfluidic chip is loaded with either 33 μl DMEM F12+Glutamax+1% FCS+1% antibiotic (streptomycin, penicillin) (FIG. 8) or 33 μl DMEM F12+Glutamax+10 μl pure FCS+1% antibiotic (streptomycin, penicillin) (FIG. 9). After 7 days of culture in an incubator with 5% CO₂ and 95% humidity photographs are taken from an inverted fluorescence microscope.

Cells are found to move towards and particularly into the reservoir under both conditions tested, with a larger quantity of cells in the presence of 10 μl of pure FCS within the reservoir.

2) MDA-MB-231 cells stably transfected with green fluorescent protein (GFP) are trypsinized at DO. Then 100 000 cells are seeded in a 50 mm diameter petri dish with the microfluidic chip placed in the center of the petri dish. The cells are cultured in Dulbecco's Modified Eagle Medium/Nutrient Mixture F-12 (DMEM F12)+Glutamax+1% fetal calf serum (FCS)+1% antibiotic (streptomycin, penicillin).

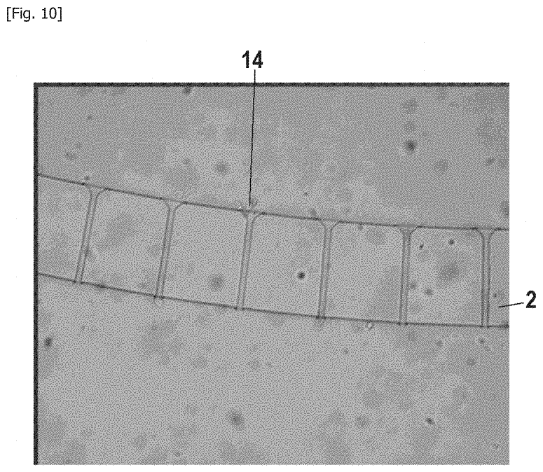

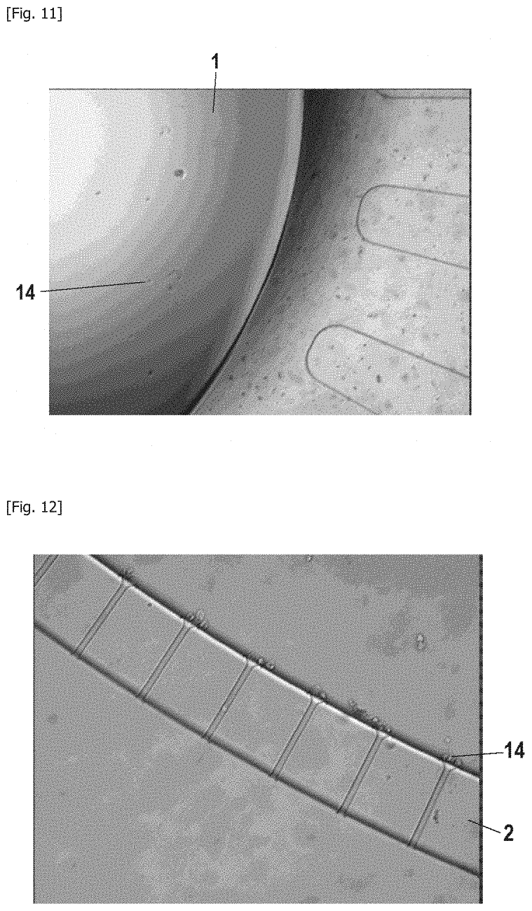

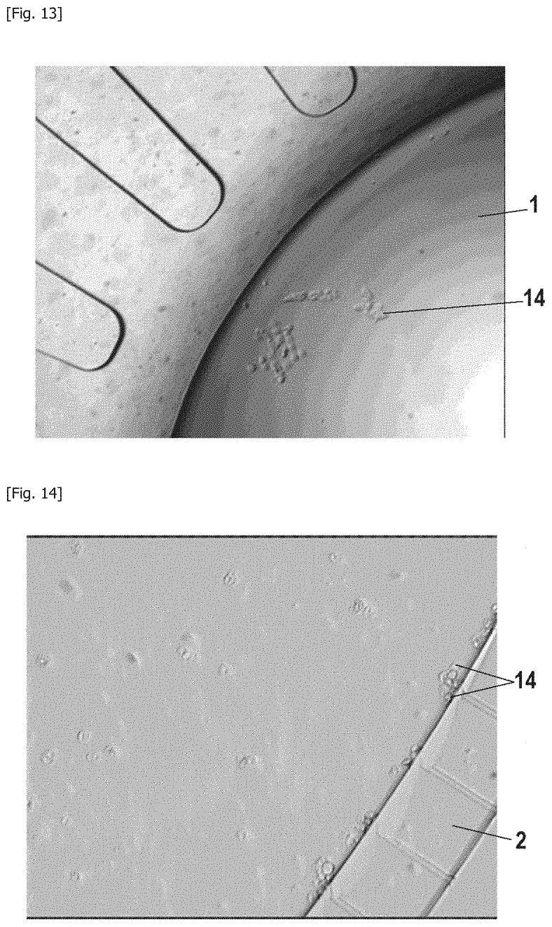

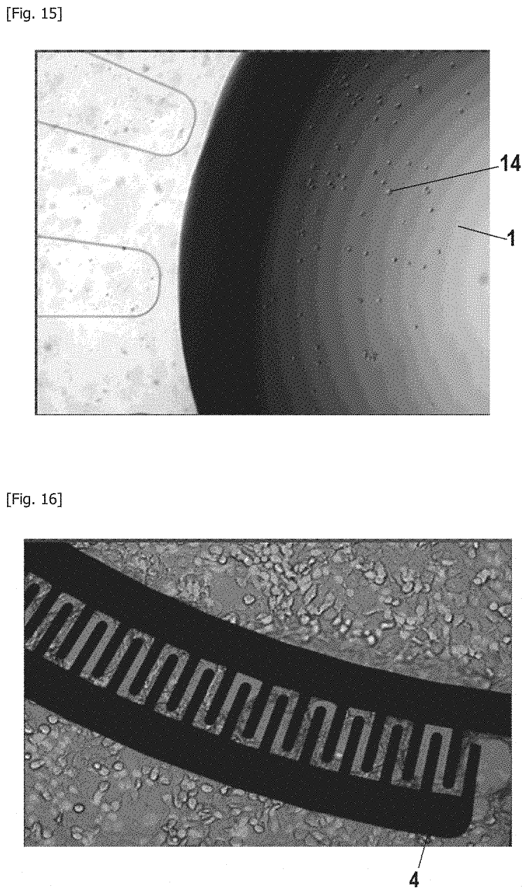

The central reservoir of the microfluidic chip is loaded with either 50 μl DMEM F12+Glutamax+1% FCS+1% antibiotic (streptomycin, penicillin) (FIGS. 10 and 11) or with 50 μl DMEM F12+Glutamax+FCS 10 μl+1% antibiotic (streptomycin, penicillin) (FIGS. 12 and 13) or with 50 μl DMEM F12+Glutamax+1 μg SDF-1+1% antibiotic (streptomycin, penicillin) (FIGS. 14 and 15).

After 8 days of culture in an incubator with 5% CO₂ and 95% humidity, photographs are taken with an inverted fluorescence microscope.

The cells are found to move towards and particularly into the reservoir under all conditions tested, particularly in the presence of a fetal calf serum gradient (FIGS. 12 and 13) and even more significantly in the presence of a compound SDF-1 gradient (FIGS. 14 and 15).

B) Ability to Destroy Cells by an Interdigitated Electrode

1) The ability of an interdigitated electrode generating an electric field to destroy MDA-MB-231 cells was tested. To this end, MDA-MB-231 cells stably transfected with green fluorescent protein (GFP) were trypsinized at DO. Then 60 000 cells were seeded in a PDMS well fixed on a glass slide inside which a circular interdigitated electrode was placed. The culture medium used is: DMEM F12+Glutamax+FCS 10%+1% antibiotic (streptomycin, penicillin).

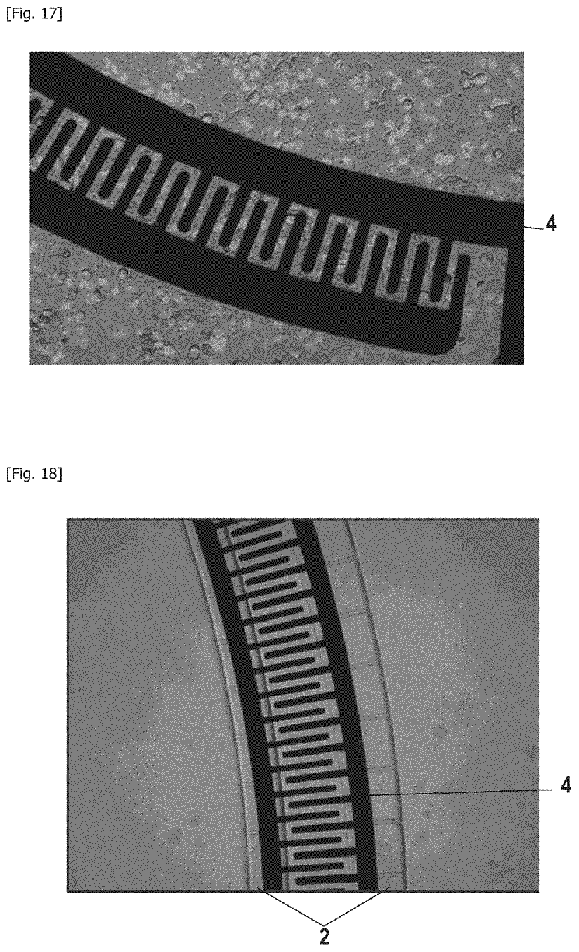

After 48 h of culture in an incubator with 5% CO₂ and 95% humidity a cell monolayer is obtained which covers the whole glass slide containing the interdigitated electrode (FIG. 16), then the cells are electroporated by application of an electric field of 5 V 100 μs 1 hertz on the slide, after 3 hours photographs are taken from an inverted fluorescence microscope (FIG. 17).

It is observed that about 70% of the cells are destroyed after 3 hours following electroporation.

2) The ability of a microfluidic chip in accordance with the invention to attract and destroy MDA-MB-231 cells by electroporation was tested.

To this end, MDA-MB-231 cells stably transfected with green fluorescent protein (GFP) are trypsinized at DO. Then 50 000 cells are seeded in a 50 mm diameter petri dish, the microfluidic chip comprising an interdigitated electrode and two networks of microchannels being placed in the center of this petri dish.

Cells are cultured in Dulbecco's Modified Eagle Medium/Nutrient Mixture F-12 (DMEM F12)+Glutamax+1% fetal calf serum (FCS)+1% antibiotic (streptomycin, penicillin).

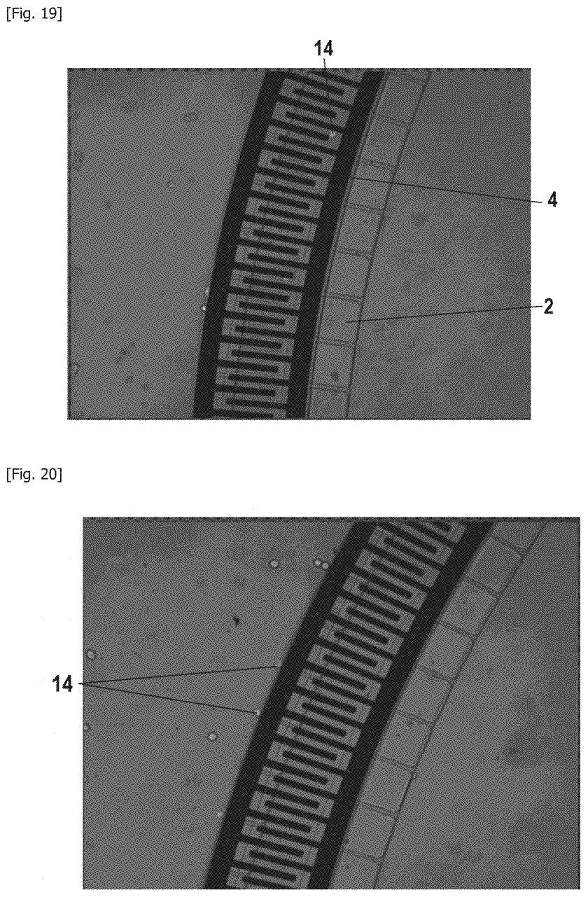

Three conditions are tested:
The central reservoir of the chip is loaded with 50 μl of DMEM F12+Glutamax+FCS 1% (fetal calf serum)+1% antibiotic (streptomycin, penicillin) in the absence of gradient and without pulsed electric field (FIG. 18)
The central reservoir of the chip is loaded with 50 μl of DMEM F12+Glutamax+SDF-11 μg+1% antibiotic (streptomycin, penicillin) without pulsed electric field (FIG. 19)
The central reservoir of the chip is loaded with 50 μl of DMEM F12+Glutamax+SDF-11 μg+1% antibiotic (streptomycin, penicillin) with pulsed electric field (FIG. 20)

After 3 days of culture, electroporations are applied in condition C every 3 hours (5 V 100 ms 1 hertz) during 7 days of culture in an incubator with 5% CO₂ and 95% humidity. Photographs are taken with an inverted fluorescence microscope.

This study demonstrated that MDA-MB-231 cells are attracted to the central reservoir and remain localized in the space located between the two networks of microchannels in the presence of the chemoattractant SDF-1 gradient and in the absence of electric field.

In the presence of the chemoattractant SDF-1 and the pulsed electric field applied every 3 hours (5 V 100 μs 1 hertz), the cells pass through the first network of microchannels and are electroporated.

In all cases, the cells are not able to cross both networks of microchannels.

These different experiments have demonstrated the ability of the microfluidic chip to attract and destroy a biological element, and in particular that said chip is able to attract breast cancer tumor cells (MDA-MB-231) either from a gradient of fetal calf serum or more significantly from a gradient of chemoattractant SDF-1 and that it is possible to destroy these same tumor cells from a pulsed electric field.

1. A microfluidic chip for attracting and destroying a specific biological element, said chip comprising:
a reservoir (1) consisting of a matrix comprising a chemoattractant compound capable of attracting the biological element
at least one network of microchannels (2) arranged between the reservoir (1) and the external environment (3) of the chip and allowing the chemoattractant compound to pass to said environment and the biological element present in said environment to pass to the reservoir (1)
at least one electrode (4) arranged between the reservoir (1) and the network of microchannels (2) or at the same location as the network of microchannels (2), said electrode being capable of generating an electric field so as to destroy the biological element during its passage to the reservoir (1).

2. The microfluidic chip according to claim 1, comprising a lower part (5) comprising a part of the reservoir (1), the network of microchannels (2) and the electrode (4), and an upper part (6) comprising a part of the reservoir (1) and suitable to be arranged on the lower part (5), said parts being fixed to each other.

3. The microfluidic chip according to claim 1, wherein the reservoir (1), the network of microchannels (2) and the electrode (4) are ring-shaped and wherein the reservoir is located in the center of the chip.

4. The microfluidic chip according to claim 2, wherein the upper part (6) and/or the lower part (5) comprise a ring-shaped cavity i) (8) capable of receiving the matrix comprising the chemoattractant compound via one or more openings (9) communicating with the external environment (3) and opening into said cavity, and a ring-shaped cavity ii) (10) arranged between the reservoir (1) and the network of microchannels (2) suitable for receiving a liquid into which the chemoattractant compound is capable of diffusing, by means of one or more openings (11) communicating with the external environment (3) and opening into said cavity.

5. The microfluidic chip according to claim 2, wherein the upper part (6) and the lower part (5) are composed of a biocompatible material.

6. The microfluidic chip according to claim 1, wherein the matrix comprising the chemoattractant compound is composed of a biocompatible material, preferably a crosslinked polymer.

7. The microfluidic chip according to claim 1, wherein the mass percentage of chemoattractant compound/matrix of the reservoir (1) is comprised between 0.1 and 20%, preferably between 0.5 and 10% and more preferably between 0.5 and 5%.

8. The microfluidic chip according to claim 1, wherein the chemoattractant compound comprised in the reservoir (1) is selected from at least one of the following compounds: CXCL12, CCL5, CCL2, CCL3, CCL7, CCL19, CCL21, CCL22, CCL25, CXCL1, CXCL5, CXCL6, CXCL8, CX3CL1, TGF alpha, TGF beta, FGF, PDGF, EGF, VEGF.

9. The microfluidic chip according to claim 1, wherein each microchannel comprised in the network of microchannels (2) has a height comprised between 1 and 40 μm, a width comprised between 1 and 40 μm, a length comprised between 30 and 250 μm.

10. The microfluidic chip according to claim 1, wherein the electrode (4) is an interdigitated electrode.

11. The microfluidic chip according to claim 1, wherein the electrode (4) is a wireless electrode said chip comprising an antenna activatable by a second antenna.

12. The microfluidic chip according to claim 1, wherein the electric field generated by the electrode (4) is capable of destroying the biological element by irreversible electroporation.

13. The microfluidic chip according to claim 1, wherein the electric field generated by the electrode (4) is a pulsed electric field comprised between 1000 V/cm and 6500 V/cm voltage, preferably between 3000 V/cm and 5000 V/cm, 1 Hz frequency and a duration comprised between 100 μs and 200 μs.
